(19) United States
(12) Patent Application Publication
(10) Pub. No.: US 2021/0097713 A1
(43) Pub. Date: Apr. 1, 2021

(54) INFORMATION PROCESSING DEVICE, INFORMATION PROCESSING METHOD, AND PROGRAM

(71) Applicant: SONY CORPORATION, TOKYO (JP)

(72) Inventors: YUSUKE NAKAMURA, KANAGAWA (JP); EISUKE NOMURA, TOKYO (JP); YUKI YAMAMOTO, CHIBA (JP); RYO OGAWA, KANAGAWA (JP)

(21) Appl. No.: 16/970,799

(22) PCT Filed: Dec. 13, 2018
(86) PCT No.: PCT/JP2018/045970

§ 371 Date: Aug. 18, 2020

(30) Foreign Application Priority Data
Feb. 27, 2018 (JP) 2018-033164

(51) Int. Cl. G06T 7/73; H04N 5/225; G06K 9/00

(52) U.S. Cl. CPC G06T 7/73; H04N 5/2254; G02B 5/30; G06T 2207/30201; G06K 9/00604

(57) ABSTRACT

[Problem] To provide an information processing device, an information processing method, and a program that enable detection, with a higher degree of accuracy, of the bright spots and the pupils from the light reflected from the eyes.

[Solution] An information processing device includes a light source that includes a first polarization filter; a sensor that includes a second polarization filter; and a control unit that processes images obtained by the sensor. The second polarization filter includes an orthogonal polarization filter having a direction perpendicular to the polarization direction of the first polarization filter, and includes a parallel polarization filter having a direction parallel to the polarization direction of the first polarization filter. The control unit detects the bright spot from a parallel polarization image obtained by the sensor, and detects the pupil from an orthogonal polarization image obtained by the sensor.

### FIELD

The application concerned is related to an information processing device, an information processing method, and a program.

### BACKGROUND

Conventionally, the corneal reflex method is widely implemented as one of the gaze detection methods. In the corneal reflex method, the eyes are irradiated with infrared light, and the gaze direction is estimated using the reflected images formed on the corneal surface and using the images formed by performing infrared imaging of the pupils.

Regarding the estimation of the gaze direction using the corneal reflex method, for example, the disclosure is given in Patent Literature 1 mentioned below.

### CITATION LIST

### Patent Literature

Patent Literature 1: International Publication Pamphlet No. 2017/013913

### SUMMARY

### Technical Problem

However, Patent Literature 1 mentioned above is related to the estimation of the gaze direction of a user wearing eyeglasses, and the object is to separate off the reflection occurring from the surface of the eyeglasses and the reflection occurring from the corneal surface.

In that regard, in the application concerned, an information processing device, an information processing method, and a program are proposed that enable detection, with a higher degree of accuracy, of the bright spots and the pupils from the light reflected from the eyes.

### Solution to Problem

According to the present disclosure, an information processing device is provided that includes: a light source that includes a first polarization filter; a sensor that includes a second polarization filter; and a control unit that processes an image obtained by the sensor, wherein the second polarization filter includes an orthogonal polarization filter having a direction perpendicular to a polarization direction of the first polarization filter, and a parallel polarization filter having a direction parallel to the polarization direction of the first polarization filter, and the control unit detects a bright spot from a parallel polarization image obtained by the sensor, and detects a pupil from an orthogonal polarization image obtained by the sensor.

According to the present disclosure, an information processing method implemented in a processor is provided that includes: obtaining a parallel polarization image and an orthogonal polarization image from a sensor that includes a second polarization filter, the second polarization filter including an orthogonal polarization filter having a direction perpendicular to a polarization direction of a first polarization filter installed in a light source, and a parallel polarization filter having a direction parallel to the polarization direction of the first polarization filter; detecting a bright spot from the parallel polarization image; and detecting a pupil from the orthogonal polarization image.

According to the present disclosure, a program is provided that causes a computer to function as a control unit to perform: an operation of obtaining a parallel polarization image and an orthogonal polarization image from a sensor that includes a second polarization filter, the second polarization filter including an orthogonal polarization filter having a direction perpendicular to a polarization direction of a first polarization filter installed in a light source, and a parallel polarization filter having a direction parallel to the polarization direction of the first polarization filter; an operation of detecting a bright spot from the parallel polarization image; and an operation of detecting a pupil from the orthogonal polarization image.

### Advantageous Effects of Invention

As described above, according to the application concerned, the bright spots and the pupils can be detected, with a higher degree of accuracy, from the light reflected from the eyes.

Meanwhile, the abovementioned effect is not necessarily limited in scope and, in place of or in addition to the abovementioned effect, any other effect indicated in the present written description or any other effect that may occur from the present written description can also be achieved.

### BRIEF DESCRIPTION OF DRAWINGS

FIG. 1 is a diagram for explaining an overview of a gaze estimation system according to an embodiment of the application concerned.

FIG. 2 is a diagram for explaining a case in which a commonplace RGB image sensor is used to take images including the first Purkinje image and the fundus reflex image.

FIG. 3 is a diagram illustrating an exemplary overall configuration of the gaze estimation system according to the embodiment.

FIG. 4 is a diagram for explaining the reflection of infrared light that has been bombarded onto an eye.

FIG. 5 is a diagram illustrating an example of arrangement of parallel polarization pixels and orthogonal polarization pixels in a polarization sensor (an imaging device) according to the embodiment.

FIG. 6 is a diagram illustrating another configuration of the gaze estimation system according to the embodiment.

FIG. 7 is a block diagram illustrating an exemplary configuration of a gaze estimation arithmetic device according to the embodiment.

FIG. 8A is a diagram for explaining about formation of a parallel polarization image according to the embodiment.

FIG. 8B is a diagram for explaining about formation of an orthogonal polarization image according to the embodiment.

FIG. 9 is a flowchart for explaining an exemplary flow of a gaze estimation operation according to the embodiment.

FIG. 10 is a schematic configuration diagram of an optical block according to a modification example of the embodiment.

FIG. 11 is a flowchart for explaining an exemplary flow of a gaze estimation operation according to the modification example of the embodiment.

FIG. 12 is a hardware configuration diagram illustrating a hardware configuration of an information processing device according to the embodiment.

### DESCRIPTION OF EMBODIMENTS

A preferred embodiment of the application concerned is described below in detail with reference to the accompanying drawings. In the present written description and the drawings, the constituent elements having practically an identical functional configuration are referred to by the same reference numerals, and the explanation is not given repeatedly.

The explanation is given in the following sequence.
1. Overview of a gaze estimation system according to an embodiment of the application concerned
2. Configuration
   2-1. System configuration
   2-2. Configuration of gaze estimation arithmetic device 100
3. Operations
4. Modification example
5. Exemplary hardware configuration
6. Summary

### 1. Overview of a Gaze Estimation System According to an Embodiment of the Application Concerned

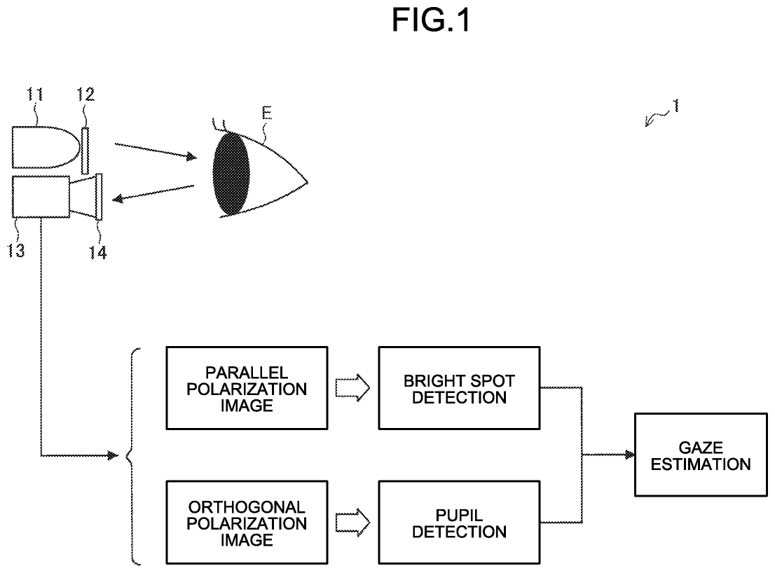

FIG. 1 is a diagram for explaining an overview of a gaze estimation system 1 (an information processing device) according to the embodiment of the application concerned. In the gaze estimation system 1 according to the present embodiment, an eye E is irradiated with infrared light after being emitted from an infrared light source 11 and polarized by a polarization filter 12; the reflected image of the infrared light is photographed by an imaging device 13; and the bright spot and the pupil that are required in estimating the gaze direction are detected from the photographed image.

### Background

Conventionally, in the bright pupil method representing one of the gaze detection methods, in order to separate the first Purkinje image and the fundus reflex light, it is necessary to make a distinction according to the sensor output of an image sensor or a photo detector (PD). However, if the reflected lights have only a small luminosity difference therebetween, the distinction cannot be made in a correct manner.

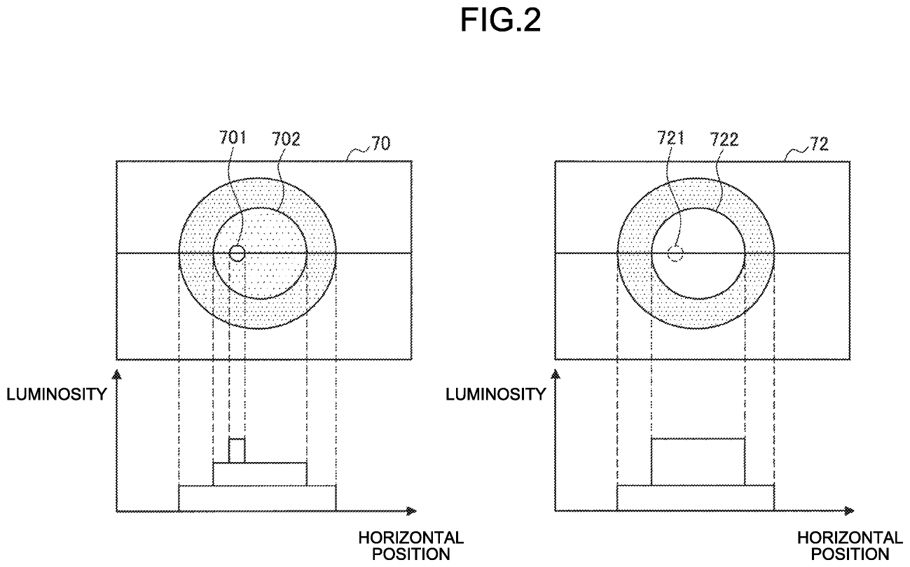

For example, explained below with reference to FIG. 2 is a case in which a commonplace RGB image sensor is used to take images including the first Purkinje image and the fundus reflex image. If the first Purkinje image has sufficiently higher luminosity as compared to the fundus reflex image, the distinction therebetween can be easily made based on the threshold value processing. That is, as illustrated in a photographed image 70 in the left-hand side portion in FIG. 2, a first Purkinje image 701 and a fundus reflex image 702 can be distinguished according to the levels of luminosity. However, as illustrated in a photographed image 72 in the right-hand side portion in FIG. 2, when a first Purkinje image 721 and a fundus reflex image 722 have comparable levels of luminosity, the separation of those two images is not possible, in principle, using the threshold value processing.

In that regard, in the gaze estimation system 1 according to the present embodiment, as illustrated in FIG. 1, the infrared light source 11 is used that includes the polarization filter 12 and that bombards infrared light onto a photographic subject; and the imaging device 13 is used that includes a polarization filter 14 including an orthogonal polarization filter having a direction perpendicular to the polarization direction of the polarization filter 12 and including a parallel polarization filter having a direction parallel to the polarization direction of the polarization filter 12. As a result, a parallel polarization image and an orthogonal polarization image can be obtained from the imaging device 13; the pupil and the bright spot to be used in gaze direction detection can be detected with more reliability; and the accuracy of a gaze estimation can be enhanced. That is, in the gaze estimation system 1 according to the present embodiment, an orthogonal polarization filter having an orthogonal relationship with the polarization filter installed in the light source and a parallel polarization filter having a parallel relationship with the polarization filter installed in the light source are installed for each pixel in the sensor; so that the two reflected lights, namely, the first Purkinje image equivalent to the bright spot and the fundus reflex light equivalent to the pupil can be separated off with more reliability. Given below is the detailed explanation of a configuration and the functions of the gaze estimation system 1 according to the present embodiment.

### 2. Configuration

### 2-1. System Configuration

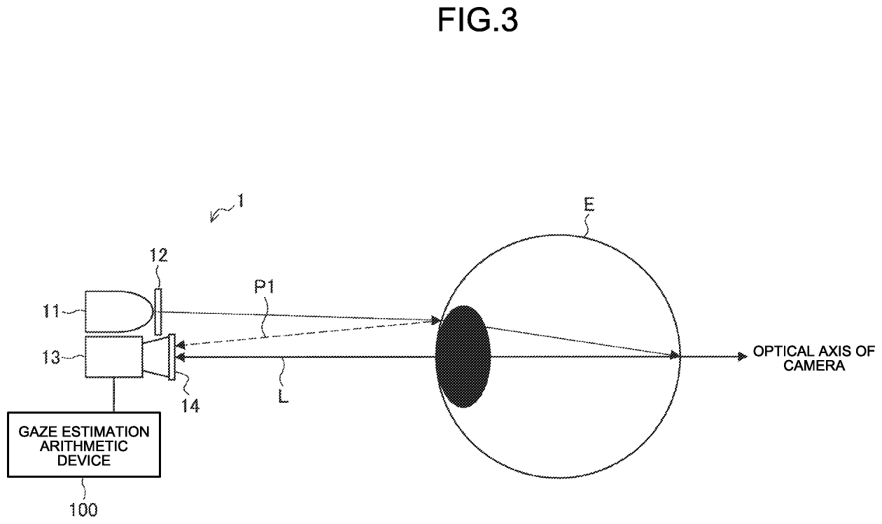

FIG. 3 is a diagram illustrating an exemplary overall configuration of the gaze estimation system 1 according to the present embodiment. As illustrated in FIG. 3, the gaze estimation system 1 (an information processing device) according to the present embodiment includes the infrared light source 11, the polarization filter 12, the imaging device 13, the polarization filter 14, and a gaze estimation arithmetic device 100. There can be at least a single infrared light source 11, at least a single polarization filter 12, at least a single imaging device 13, and at least a single polarization filter 14. The images taken by the imaging device 13 are output to the gaze estimation arithmetic device 100 that estimates the gaze direction.

### Infrared Light Source 11

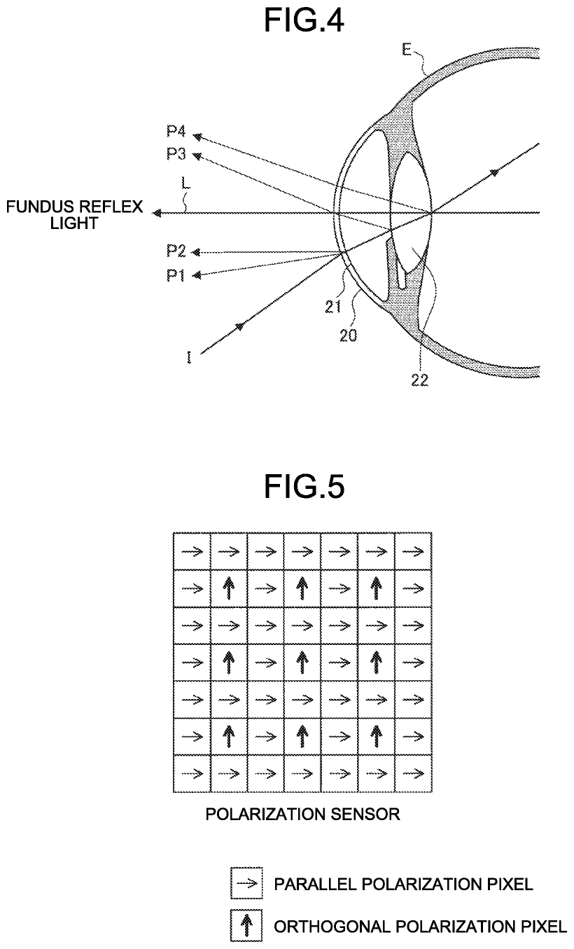

The infrared light source 11 is a light source that bombards infrared light onto the eye E for the purpose of obtaining corneal reflection; and can be, for example, an infrared LED. In the infrared light source 11, the polarization filter 12 is installed. Hence, the infrared light that has been polarized by the polarization filter 12 gets bombarded onto the eye E. Regarding the reflection of infrared light that is bombarded onto an eye, the explanation is given below with reference to FIG. 4. As illustrated in FIG. 4, when near-infrared light I is bombarded onto a corneal surface 20, it gets separated off into the light reflected from the corneal surface 20 and the light entering inside the eye from the cornea. More accurately, the near-infrared light I gets separated off into the following: the light reflected from the corneal surface 20 (the first Purkinje image P1); the light reflected from a corneal posterior 21 (the second Purkinje image P2); the light reflected from the anterior surface of a crystalline lens 22 (the third Purkinje image P3); the light reflected from the posterior surface of the crystalline lens 22 (the fourth Purkinje image P4); and the light reflected from the ocular fundus (a fundus reflex light L). Usually, in the near-infrared light having the quantity of light in the order of mW, it is known that the light intensity of the second Purkinje image to the fourth Purkinje image is not sufficient and is considered almost negligible. Thus, in the present embodiment, the first Purkinje image P1 and the fundus reflex light L are used in performing the gaze estimation.

### Imaging Device 13

The imaging device 13 is a device for photographing the eye E that is irradiated with infrared light. In the imaging device 13, the polarization filter 14 is installed, and imaging of two polarization directions can be simultaneously performed. Herein, polarized light implies the light that oscillates only in the directions having specific electric field and specific magnetic field. In the measurement performed in the imaging device 13, the polarized light in specific directions is transmitted/absorbed using the polarization filter 14, and imaging of that light is performed. Meanwhile, since images in the infrared region are used in the gaze estimation, a device capable of performing imaging of the infrared region is used as the imaging device 13.

The polarization filter 14 installed in the imaging device 13 includes an orthogonal polarization filter having a direction perpendicular to the polarization direction of the polarization filter 12 installed in the light source, and includes a parallel polarization filter having a direction parallel to the polarization direction of the polarization filter 12. The orthogonal polarization filter and the parallel polarization filter are installed for each pixel in the imaging device 13. In the present written description, the pixels for which the orthogonal polarization filter is installed are called “orthogonal polarization pixels,” and the pixels for which the parallel polarization filter is installed are called “parallel polarization pixels.” Moreover, in the present written description, the imaging device 13 including the polarization filter 14 is also called “a polarization sensor.”

In the present embodiment, a method is provided by which, of the light reflecting from the eye E, the first Purkinje image P1 and the fundus reflex light L are separated off using the polarization. More particularly, the separation is performed according to the principle explained below.

When the reflection occurs at the corneal surface, the first Purkinje image P1 has its polarization maintained and falls on the sensor surface in the polarized state. Hence, the first Purkinje image P1 can be detected with the parallel polarization pixels having a parallel relationship with the polarization direction of the polarization filter 12 of the infrared light source 11. In contrast, the fundus reflex light L scatters inside the eye thereby losing its polarized state, and thus falls on the sensor surface in the depolarized state. Hence, the fundus reflex light L can be detected with the orthogonal polarization pixels having an orthogonal relationship with the polarization direction of the polarization filter 12.

In FIG. 5 is illustrated an example of an arrangement of the parallel polarization pixels and the orthogonal polarization pixels in a polarization sensor. Generally, the bright spot (the first Purkinje image P1) that is obtained as a result of the reflection from the corneal surface is imaged on the imaging surface in a sufficiently smaller size as compared to the size of the pupil (the fundus reflex light L). Hence, the detection of the bright spot requires higher resolution as compared to the detection of the pupil. Thus, as illustrated in FIG. 5, it is desirable to have such a configuration of the orthogonal polarization pixels and the parallel polarization pixels that the number of parallel polarization pixels is greater than the number of orthogonal polarization pixels. Although there is no particular restriction on the method of arranging the polarization pixels; for example, the orthogonal polarization pixels that are arranged distantly from each other can be individually surrounded by a plurality of parallel polarization pixels as illustrated in FIG. 5. That is, for example, the polarization sensor can be configured in such a way that each orthogonal polarization pixel is surrounded by eight parallel polarization pixels.

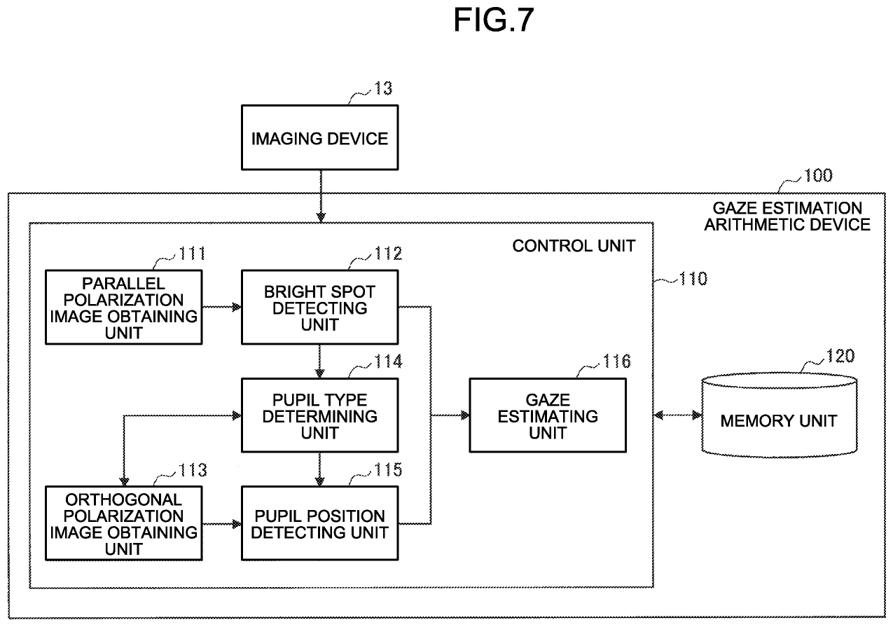

An image obtained by imaging by the imaging device 13 is then output to the gaze estimation arithmetic device 100 that estimates the gaze direction. In the gaze estimation arithmetic device 100, from the image input thereto, the first Purkinje image P1 (the light reflected from the cornea) that fell on the imaging device 13 is distinguished, with more reliability, from the fundus reflex light L (the light reflected from the fundus) that fell on the imaging device 13; and thus enhancement in the gaze estimation accuracy is achieved. A specific configuration of the gaze estimation arithmetic device 100 is explained later with reference to FIG. 7.

Regarding the positional relationship between the gaze estimation system 1 according to the present embodiment and the eye E; as long as the corneal reflection of the infrared light, which is emitted from the infrared light source 11, falls on the imaging device 13, any arrangement serves the purpose. For example, as illustrated in FIG. 3, the infrared light source 11 including the polarization filter 12 as well as the imaging device including the polarization filter 14 can be disposed in close vicinities of the eye E. Such a configuration can be implemented, for example, in an eyewear-type terminal or a head-mount device which, when worn by a user, has a lens positioned in front of the eyes of the user.

Alternatively, the infrared light source 11 including the polarization filter 12 as well as the imaging device including the polarization filter 14 can be disposed at distant positions from the eye E. Such a configuration can be implemented, for example, in a stationary terminal such as the display of a television set or a personal computer that is positioned distantly from the eyes.

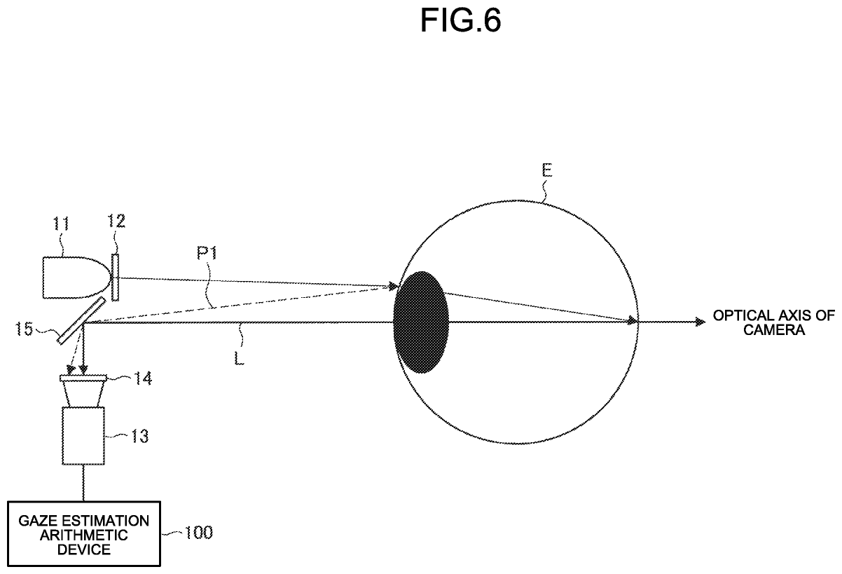

Still alternatively, for example, as illustrated in FIG. 6, a light path separating device 15 such as a half mirror can be installed in between the eye E and the imaging device 13.

Meanwhile, the configuration of the gaze estimation system 1 according to the present embodiment is not limited to the configuration explained above. That is, as long as a polarized light is bombarded onto the eye and polarization images in two directions can be simultaneously taken, it serves the purpose. Moreover, the devices in which the gaze estimation system 1 is implementable are also not limited to the examples given earlier. For example, the gaze estimation system 1 can alternatively be configured as a device that is detachably-attachable to an eyewear-type terminal.

### 2-2. Gaze Estimation Arithmetic Device 100

FIG. 7 is a block diagram illustrating an exemplary configuration of the gaze estimation arithmetic device 100 according to the present embodiment. As illustrated in FIG. 7, the gaze estimation arithmetic device 100 includes a control unit 110 and a memory unit 120.

The control unit 110 functions as an arithmetic processing device and a control device, and comprehensively controls the operations in the gaze estimation arithmetic device 100 according to various programs. The control unit 110 is implemented using, for example, an electronic circuit such as a CPU (Central Processing Unit) or a microprocessor. Moreover, the control unit 110 can include a ROM (Read Only Memory) that is used to store programs and operation parameters to be used, and a RAM (Random Access Memory) that is used to temporarily store parameters that undergo appropriate changes.

Furthermore, the control unit 110 according to the first embodiment functions as a parallel polarization image obtaining unit 111, a bright spot detecting unit 112, an orthogonal polarization image obtaining unit 113, a pupil type determining unit 114, a pupil position detecting unit 115, and a gaze estimating unit 116.

### Parallel Polarization Image Obtaining Unit 111

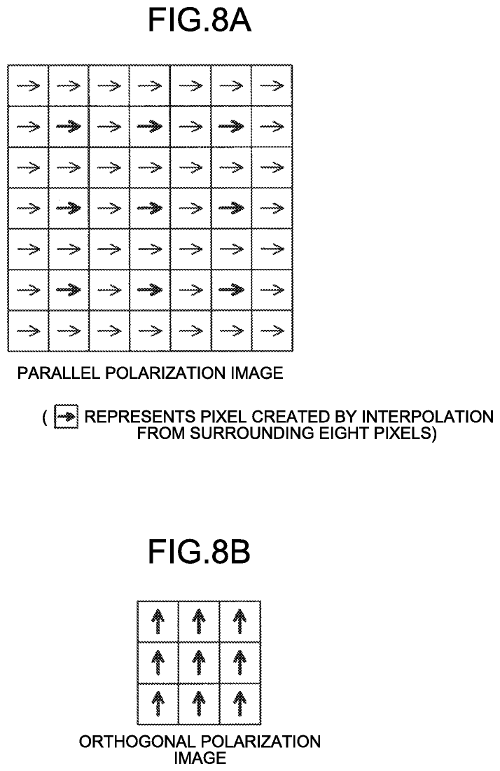

The parallel polarization image obtaining unit 111 synthesizes, as a parallel polarization image, a single image from the parallel polarization pixels of the imaging device 13 (the polarization sensor) (see FIG. 5). Herein, it is assumed that the positions of the parallel polarization pixels are internally stored as prior information in the ROM (the memory unit 120). Regarding the image size of a parallel polarization image, Nₚₕ represents the horizontal size and Nₚᵥ represents the vertical size. Herein, the parallel polarization image obtaining unit 111 synthesizes an image only from the parallel polarization pixels. However, regarding the defective pixels among the orthogonal polarization pixels, the parallel polarization image obtaining unit 111 creates pixels by interpolation from the surrounding pixels as illustrated in FIG. 8A.

### Bright Spot Detecting Unit 112

The bright spot detecting unit 112 detects the bright spot from the parallel polarization image obtained by the parallel polarization image obtaining unit 111. The position of the bright spot can be detected, for example, according to a method based on machine-learning; or such an area can be detected as the bright spot which has a higher luminosity value than the surrounding area, which has the size to be equal to or smaller than a predetermined value, and which has its detection position to be at a predetermined level of coherency or more with the installation position of the infrared light source 11. In the present written description, the bright spot corresponds to the first Purkinje image. However, the detection method is not limited to the methods explained herein.

The bright spot detecting unit 112 transforms the detected bright spot center position to relative coordinates (expressed between 0 and 1) that are normalized with the image size of the parallel polarization image. More particularly, in the case of the arrangement illustrated in FIG. 5, if (P_{gh}, P_{gv}) represents the detected bright spot center position (absolute coordinates), relative coordinates (p_{gh}, p_{gv}) are calculated according to the equation given below.

P_{gh}=(P_{gh}+0.5)/Nₚₕ

P_{gv}=(P_{gv}+0.5)/Nₚᵥ

### Orthogonal Polarization Image Obtaining Unit 113

The orthogonal polarization image obtaining unit 113 synthesizes, as an orthogonal polarization image, a single image from the orthogonal polarization pixels of the imaging device 13 (the polarization sensor) (see FIG. 5). An example of the synthesized orthogonal polarization image is illustrated in FIG. 8B. Herein, it is assumed that the positions of the orthogonal polarization pixels are internally stored as prior information in the ROM (the memory unit 120). Regarding the image size of an orthogonal polarization image, Nₛₕ represents the horizontal size and Nₛᵥ represents the vertical size. Herein, the orthogonal polarization image obtaining unit 113 synthesizes an image only from the orthogonal polarization pixels. However, if the orthogonal polarization pixels are simply stuck together, it is possible to think of a case in which the jaggy becomes conspicuous. In such a case, it is desirable to perform smoothing using a low-pass filter.

### Pupil Type Determining Unit 114

The pupil type determining unit 114 has the function of determining the phenomenon of a bright pupil/a dark pupil. The following explanation is given about the phenomenon of a bright pupil/a dark pupil. A light source of near-infrared light is disposed near the aperture of a camera (the infrared light source 11 is disposed substantially in front of the eye E) and photographing is performed by irradiating the eye with light along the optical axis of the camera (see FIG. 3), so that the light reaches the fundus from the pupil; gets reflected from the fundus; and returns to the camera aperture through the crystalline lens and the cornea. At that time, the pupil is brightly photographed, and the phenomenon is called a bright pupil. On the other hand, when photographing is performed by irradiating the eye with light from a light source that is placed at a distance from the camera aperture, the light reflected from the fundus barely falls on the camera aperture. Consequently, the pupil is darkly photographed, and the phenomenon is called a dark pupil. In the present embodiment, regarding the pupil, although it is assumed that the bright pupil is obtained, it is also believed that the dark pupil is obtained if there is a significant movement of the pupil position thereby resulting in changes in the positional relationship between the infrared light source 11, the imaging device 13, and the pupil position. In that regard, in the present embodiment, the pupil type determining unit 114 is used to determine whether the bright pupil or the dark pupil is obtained and, if the dark pupil is obtained, pupil detection is made possible by performing predetermined processing on the orthogonal polarization image.

Based on the orthogonal polarization image and based on the bright spot detection result obtained by the bright spot detecting unit 112, the pupil type determining unit 114 refers to the luminosity distribution of the pixels surrounding the bright spot position and determines whether the bright pupil or the dark pupil is obtained. More specifically, the pupil type determining unit 114 creates a luminosity profile in the horizontal direction and the vertical direction and passing through the bright spot center position; and determines that the bright pupil is obtained if the profile has an uneven shape, or determines that the dark pupil is obtained if the profile has a convex shape. However, the determination method is not limited to the method explained herein. Alternatively, for example, a discriminator based on machine-learning can be used. If the dark pupil is obtained, the pupil type determining unit 114 inverts the luminosity of the orthogonal polarization image, so that the pupil position detecting unit 115 (described below) becomes able to detect the position of the pupil (the boundary of the pupil and the pupil center position) in an identical manner to the case in which the bright pupil is obtained.

### Pupil Position Detecting Unit 115

The pupil position detecting unit 115 detects the pupil from an orthogonal polarization image. In the present written description, the pupil position corresponds to a fundus reflex image. For example, the pupil position can be detected by a method based on machine-learning; or the area that is elliptical and bright can be detected as the pupil. However, the detection method is not limited to the methods mentioned herein.

Then, the pupil position detecting unit 115 transforms the detected pupil center position to relative coordinates (expressed between 0 and 1) that are normalized with the image size of the orthogonal polarization image. More particularly, in the case of the arrangement illustrated in FIG. 5, if (Pₚₕ, Pₚᵥ) represents the detected pupil center position (absolute coordinates), relative coordinates (pₚₕ, pₚₘ) are calculated according to the equation given below.

Pₚₕ=(Pₚₕ+0.5)/Nₛₕ

Pₚᵥ=(Pₚᵥ+0.5)/Nₛᵥ

### Gaze Estimating Unit 116

The gaze estimating unit 116 estimates gaze information from the bright spot center position (p_{gh}, p_{gv}) and the pupil center position (pₚₕ, pₚᵥ). For example, when the installation positions of the infrared light source 11 and the imaging device 13 are known; the gaze estimating unit 116 estimates three-dimensional corneal-curvature-radius central coordinates from the corneal reflection image in the observed image. The gaze estimating unit 116 estimates the three-dimensional pupil central coordinates from the corneal-curvature-radius central coordinates and the pupil position in the image, and obtains the optical axis of the eye as the axis joining the two positions. Then, the gaze estimating unit 116 obtains a three-dimensional gaze vector meant for transforming the optical axis obtained from the observed information to the visual axis equivalent to the gaze direction of the person. Alternatively, the gaze estimating unit 116 can obtain the gaze vector by mapping the two-dimensional vector, which joins the corneal reflection image in the image and the pupil, with the gaze position on the display. Meanwhile, the gaze estimating unit according to the present embodiment is not limited to the explanation given herein, and alternatively various known gaze estimating methods can be implemented.

### The Memory Unit 120

The memory unit 120 is implemented using a ROM (Read Only Memory) that is used to store programs and operation parameters to be used in the operations of the control unit 110, and a RAM (Random Access Memory) that is used to temporarily store parameters that undergo appropriate changes.

Till now, the specific explanation was given about a configuration of the gaze estimation arithmetic device 100 according to the embodiment of the application concerned. However, the configuration of the gaze estimation arithmetic device 100 is not limited to the example illustrated in FIG. 7. For example, alternatively, the configuration may not include the pupil type determining unit 114; or the operations of the control unit 110 of the gaze estimation arithmetic device 100 can be performed across a plurality of devices. Meanwhile, the gaze estimation arithmetic device 100 can also control the bombardment of the infrared light from the infrared light source 11.

### 3. Operations

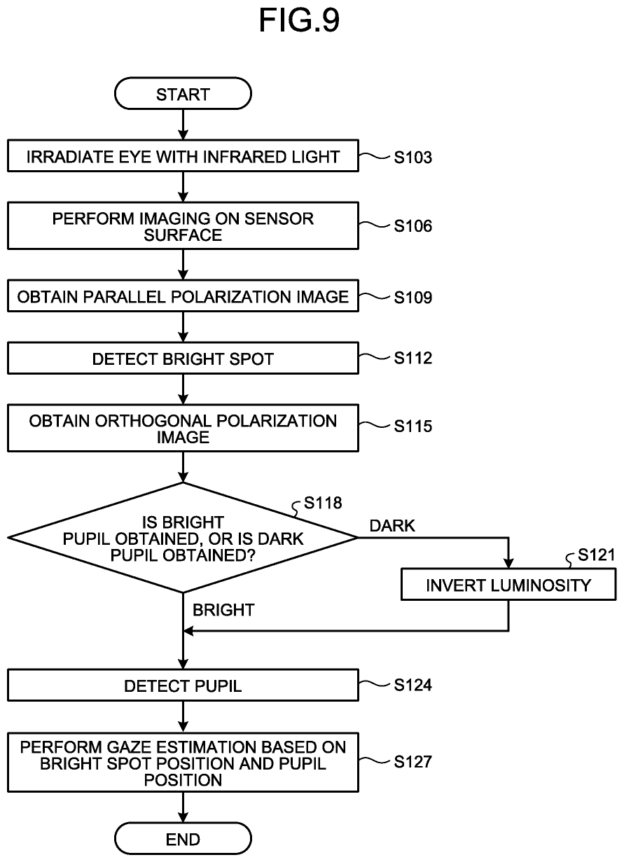

Regarding the operations performed in the gaze estimation system according to the present embodiment, the specific explanation is given below with reference to FIG. 9. FIG. 9 is a flowchart for explaining an exemplary flow of a gaze estimation operation according to the present embodiment. As illustrated in FIG. 9, firstly, in the gaze estimation system 1, the eye E is irradiated with infrared light emitted from the infrared light source 11 (Step S103).

Then, in the gaze estimation system 1, imaging of the eye E is performed on the sensor surface (by the imaging device 13 including the polarization filter 14) (Step S106).

Subsequently, in the gaze estimation arithmetic device 100, the parallel polarization image obtaining unit 111 obtains a parallel polarization image (Step S109).

Then, in the gaze estimation arithmetic device 100, the bright spot detecting unit 112 detects the bright spot from the parallel polarization image (Step S112).

Subsequently, in the gaze estimation arithmetic device 100, the orthogonal polarization image obtaining unit 113 obtains an orthogonal polarization image (Step S115).

Then, in the gaze estimation arithmetic device 100, the pupil type determining unit 114 determines whether the bright pupil or the dark pupil is obtained (Step S118).

If the dark pupil is obtained (dark at Step S118), the gaze estimation arithmetic device 100 inverts the luminosity of the orthogonal polarization image (Step S121).

Then, the gaze estimation arithmetic device 100 detects the pupil from the orthogonal polarization image (or from the orthogonal polarization image having the inverted luminosity) (Step S124).

Subsequently, the gaze estimation arithmetic device 100 performs the gaze estimation based on the detected bright spot position and the detected pupil position (Step S127).

Till now, the explanation was given about an example of the operations according to the present embodiment. The operations illustrated in FIG. 9 are only exemplary, and the application concerned is not limited to the example illustrated in FIG. 9. For example, in the application concerned, the sequence of the operations is not limited to the steps illustrated in FIG. 9. Thus, at least some of the steps can be performed in parallel; or the steps can be performed in reverse order. For example, the operations from Step S109 to Step S112 and the operations from Step S115 to Step S124 can be performed in parallel or in reverse order.

Moreover, all operations illustrated in FIG. 9 need not be performed at all times. For example, the operations from Step S118 to Step S121 may be skipped.

Furthermore, all operations illustrated in FIG. 9 need not be always performed in a single device.

Meanwhile, although not illustrated in FIG. 9, while detecting the bright spot (Step S112) and detecting the pupil (Step S124), the bright spot center position and the pupil center position can be respectively transformed to relative coordinates normalized with the image size.

### Effects

As described above, in the present embodiment, the polarization filter is configured to have higher resolution with respect to the bright spot that is smaller than the pupil, thereby enabling detection of the bright spot position with a higher degree of accuracy. Moreover, even when the bright pupil is not obtained on account of a misalignment of the light source and the camera position with respect to the corneal center position; as a result of determining whether the bright pupil is obtained or the dark pupil is obtained, the bright spot and the pupil position can be captured with the same configuration.

### 4. Modification Example

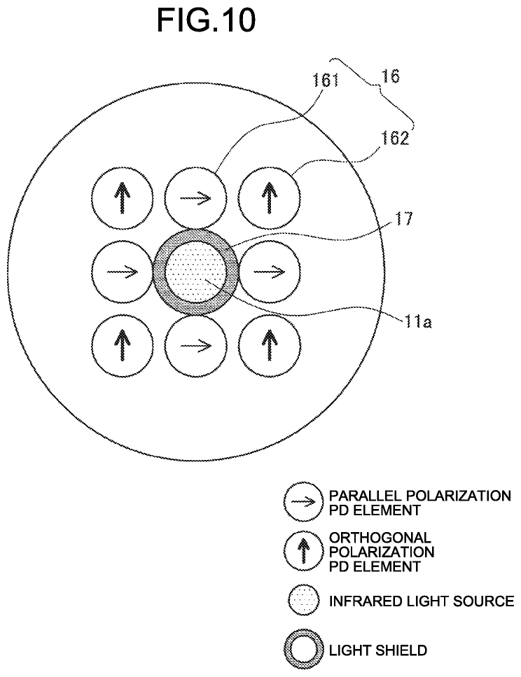

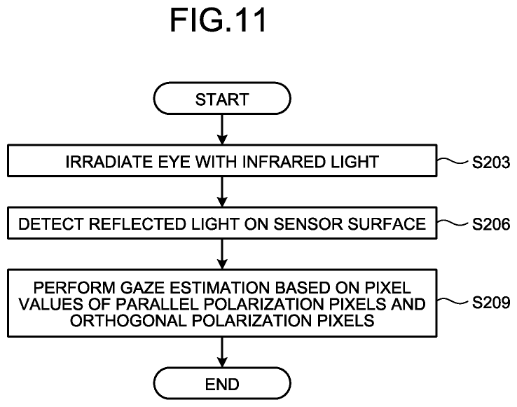

Explained below with reference to FIGS. 10 and 11 is a modification example of the gaze estimation system according to the present embodiment.

### Configuration

FIG. 10 is a schematic configuration diagram of an optical block according to the modification example of the present embodiment. The gaze estimation system according to the present modification example includes an optical block that includes: an infrared light source 11a (an infrared light emitting unit) including a polarization filter; and PD (Photo Detector) elements 16 (reflected-light detecting units) that include a polarization filter and that detect reflected light. Each PD element 16 includes a parallel polarization PD element 161 having a parallel relationship with the polarization direction of the polarization filter of the infrared light source 11a, and includes an orthogonal polarization PD element 162 having an orthogonal relationship with the polarization direction of the polarization filter of the infrared light source 11a.

In the present modification example, as illustrated in FIG. 10, the infrared light source 11a is placed in the center of the optical block (i.e., the PD elements 16 are placed around the infrared light source 11a), so that the bright pupil is inevitably obtained. Although not illustrated in FIG. 10, the polarization filter of the infrared light source 11a is placed on the anterior side of the infrared light source 11a. Moreover, the PD elements 16 placed around the infrared light source 11a are so configured that the number of parallel polarization PD elements 161 is greater than the number of orthogonal polarization PD elements 162. That is, in the present modification example, although the bright spot position is not calculated, in order to ensure that the pixel values (the luminosity distribution) obtained from the parallel polarization pixels and the orthogonal polarization pixels are of a higher degree of accuracy; generally the parallel polarization pixels, which detect the bright spot (the first Purkinje image P1) that has a sufficiently smaller size than the size of the pupil (the fundus reflex light L), can be set to have higher resolution.

Meanwhile, in order to ensure that the light emitted from the infrared light source 11a does not directly fall on the PD elements 16, it is desirable to dispose a light shield 17 in between the infrared light source 11a and the PD elements 16 as illustrated in FIG. 10. For example, the infrared light source 11a can be covered with a tubular member formed with a material having excellent light shielding properties.

Meanwhile, the number of PD elements 16 and the arrangement thereof is not limited to the example illustrated in FIG. 10.

In an identical manner to the embodiment described above, the optical block is disposed substantially in front of the eye E, and the infrared light emitted from the infrared light source 11a gets reflected from the corneal surface of the eye E and falls on the PD elements 16 while passing close to the optical center of the optical block. In the reflection from the corneal surface, since the polarization direction is maintained, the reflection can be captured only with the parallel polarization PD elements 161. On the other hand, the light that enters inside the eye without getting reflected from the corneal surface scatters inside the eye and then falls on the PD elements while passing close to the optical center of the optical block. In that case, since the polarization direction is not maintained, the light can be captured with the orthogonal polarization PD elements 162. Meanwhile, in order to enhance the gaze estimation accuracy (described later), it is desirable that the PD elements 16 have a wider dynamic range.

Meanwhile, the information about the parallel polarization pixels and the orthogonal polarization pixels as obtained by the PD elements 16 (the reflected-light detecting units) is output to the gaze estimation arithmetic device 100.

The gaze estimation arithmetic device 100 receives an input of the information about the parallel polarization pixels and the orthogonal polarization pixels as obtained by the PD elements 16 (the reflected-light detecting units), and estimates a direct gaze vector. In the preparation stage, the gaze estimation arithmetic device 100 can obtain, in advance, the luminosity value of each parallel polarization pixel and each orthogonal polarization pixel (i.e., the luminosity distribution) and a gaze vector representing the correct solution at that time; and can learn such information using a DNN (Deep Neural Network). Alternatively, the gaze estimation arithmetic device 100 can obtain the learning result in advance. Then, the gaze estimation arithmetic device 100 estimates a gaze vector using the learnt network configuration, and thus can calculate a direct gaze vector from the pixel values of the parallel polarization pixels and the orthogonal polarization pixels.

Meanwhile, the estimation method explained above is only exemplary, and some other method such as regression analysis can also be used.

Moreover, herein, a direct gaze vector is calculated from the pixel values of the parallel polarization pixels and the orthogonal polarization pixels. However, alternatively, a gaze vector representing the correct solution is learnt from the pixel values of either the parallel polarization pixels or the orthogonal polarization pixels, so that a direct gaze vector can be calculated from the pixel values of either the parallel polarization pixels or the orthogonal polarization pixels.

In the present modification example, the gaze estimation arithmetic device 100 can calculate the gaze vector directly from the output of the PD elements 16, without having to perform bright spot detection and pupil detection. Hence, in the configuration illustrated in FIG. 7, as long as the control unit 110 has the function of at least the gaze estimating unit 116, it serves the purpose.

### Operations

Explained below with reference to FIG. 11 are the operations performed in the abovementioned configuration according to the present modification example. FIG. 11 is a flowchart for explaining an exemplary flow of a gaze estimation operation according to the present modification example.

As illustrated in FIG. 11, firstly, in the gaze estimation system according to the present modification example, the eye E is irradiated with infrared light emitted from the infrared light source 11a (Step S203).

Then, in the gaze estimation system, the reflected light is detected on the sensor surface (by the PD elements 16 including a polarization filter) (Step S106).

Then, the gaze estimation arithmetic device 100 performs the gaze estimation from the pixel values (the luminosity distribution) of the parallel polarization pixels and the orthogonal polarization pixels (Step S209).

### Effect

As described above, in the gaze estimation system according to the present modification example, the gaze vector can be calculated directly from the output of the PD elements without having to perform bright spot detection and pupil detection. That enables achieving reductions in the implementation cost.

### 5. Exemplary Hardware Configuration

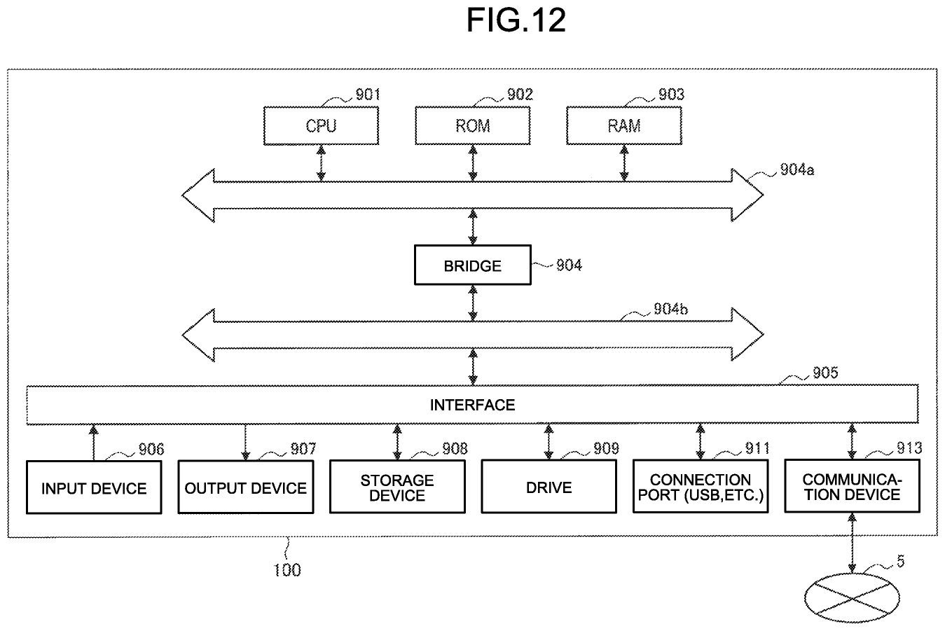

Lastly, the explanation is given about an exemplary hardware configuration of the gaze estimation arithmetic device 100 according to the present embodiment. FIG. 12 is a hardware configuration diagram illustrating a hardware configuration of the gaze estimation arithmetic device 100 according to the present embodiment.

The gaze estimation arithmetic device 100 according to the present embodiment can be implemented using a processing device such as a computer. As illustrated in FIG. 11, the gaze estimation arithmetic device 100 includes a CPU (Central Processing Unit) 901, a ROM (Read Only Memory) 902, a RAM (Random Access Memory) 903, and a host bus 904a. Moreover, the gaze estimation arithmetic device 100 includes a bridge 904, an external bus 904b, an interface 905, an input device 906, an output device 907, a storage device 908, a drive 909, a connection port 911, and a communication device 913.

The CPU 901 functions as an arithmetic processing device and a control device, and comprehensively controls the operations in the gaze estimation arithmetic device 100 according to various programs. The CPU 901 can be a microprocessor too. The ROM 902 is used to store the programs and the operation parameters to be used by the CPU 901. The RAM 903 is used to temporarily store the programs during their execution by the CPU 901, and to temporarily store the parameters that undergo appropriate changes during the execution of the programs. These constituent elements are connected to each other by the host bus 904a that is configured using a CPU bus.

The host bus 904a is connected to the external bus 904b such as a PCI (Peripheral Component Interconnect/Interface) bus. Meanwhile, the host bus 904a, the bridge 904, and the external bus 904b need not always be configured separately, and alternatively the functions of those buses can be implemented in a single bus.

The input device 906 is configured using an input unit such as a mouse, a keyboard, a touch-sensitive panel, buttons, a microphone, switches, or levers for enabling the user to input information; and an input control circuit that generates input signals based on the user input and outputs the input signals to the CPU 901. The output device 907 includes, for example, a display device such as a liquid crystal display (LCD) device, an OLED (Organic Light Emitting Diode) device, and a lamp; and includes a sound output device such as a speaker.

The storage device 908 is an example of the memory unit of the gaze estimation arithmetic device 100, and is used to store data. The storage device 908 can include a memory medium, a recording device for recording data in the memory medium, a reading device for reading data from the memory medium, and a deleting device for deleting the data recorded in the memory medium. The storage device 908 drives a hard disk, and stores programs to be executed by the CPU 901 and stores a variety of data.

The drive 909 is a reader/writer with respect to memory mediums, and is either embedded in the gaze estimation arithmetic device 10 or attached to the outside. The drive 909 reads information that is recorded in a removable recording medium such as a magnetic disk, an optical disk, a magneto-optical disk, or a semiconductor memory that is inserted; and outputs the read information to the RAM 903.

The connection port 911 is an interface for establishing connection with external devices, and functions as a connection port for external devices capable of data transmission using, for example, the USB (Universal Serial Bus). The communication device 913 is a communication interface configured using, for example, a communication device meant for establishing communication with a network 5. Herein, the communication device 913 can be a communication device compatible to a wireless LAN (Local Area Network), or can be a communication device compatible to a wireless USB, or can be a wired communication device performing wired communication.

### 5. Summary

As described above, in the gaze estimation system according to the embodiment of the application concerned, it becomes possible to detect, with a higher degree of accuracy, the bright spot and the pupil from the light reflected from the eye.

Although the application concerned is described above in detail in the form of an embodiment with reference to the accompanying drawings; the technical scope of the application concerned is not limited to the embodiment described above. That is, the application concerned is to be construed as embodying all modifications such as other embodiments, additions, alternative constructions, and deletions that may occur to one skilled in the art that fairly falls within the basic teaching herein set forth. In any form thereof, as long as the functions/effects of the application concerned are achieved, the modifications are included in the scope of the application concerned.

For example, a computer program for implementing the functions of the gaze estimation arithmetic device 100 can be created in the hardware such as the CPU, the ROM, and the RAM embedded in the gaze estimation arithmetic device 100. Moreover, a computer-readable memory medium having that computer program stored can also be provided.

The effects described in the present written description are only explanatory and exemplary, and are not limited in scope. That is, in addition to or in place of the effects described above, the technology disclosed in the application concerned enables achieving other effects that may occur to one skilled in the art.

Meanwhile, a configuration as explained below also falls within the technical scope of the application concerned.

(1)

An information processing device comprising:

a light source that includes a first polarization filter;

a sensor that includes a second polarization filter; and

a control unit that processes an image obtained by the sensor, wherein

the second polarization filter includes
an orthogonal polarization filter having a direction perpendicular to a polarization direction of the first polarization filter, and
a parallel polarization filter having a direction parallel to the polarization direction of the first polarization filter, and

the control unit
detects a bright spot from a parallel polarization image obtained by the sensor, and
detects a pupil from an orthogonal polarization image obtained by the sensor.
   (2)

The information processing device according to (1), wherein the control unit estimates gaze information based on a center position of the pupil and a center position of the bright spot.

(3)

The information processing device according to (2), wherein the control unit

detects, as the bright spot, a first Purkinje image from the parallel polarization image, and

detects, as the pupil, fundus reflex light from the orthogonal polarization image.

(4)

The information processing device according to (3), wherein, from among pixels of the sensor, the number of parallel polarization pixels corresponding to the parallel polarization filter is greater than the number of orthogonal polarization pixels corresponding to the orthogonal polarization filter.

(5)

The information processing device according to (4), wherein pixels of the sensor are formed with an arrangement in which the orthogonal polarization pixels, which are disposed distantly, are individually surrounded by a plurality of the parallel polarization pixels.

(6)

The information processing device according to any one of (2) to (5), wherein the control unit

transforms a center position of the detected bright spot to relative coordinates normalized with an image size of the parallel polarization image,

transforms a center position of the detected pupil to relative coordinates normalized with an image size of the orthogonal polarization image, and

estimates the gaze information based on each set of the normalized relative coordinates.

(7)

The information processing device according to any one of (1) to (6), wherein the control unit

determines, based on a bright spot detection result obtained from the orthogonal polarization image and the parallel polarization image, whether a bright pupil or a dark pupil is obtained, and

when a dark pupil is obtained, inverts luminosity of the orthogonal polarization image and then detects the pupil.

(8)

An information processing method implemented in a processor, comprising:

obtaining a parallel polarization image and an orthogonal polarization image from a sensor that includes a second polarization filter, the second polarization filter including
an orthogonal polarization filter having a direction perpendicular to a polarization direction of a first polarization filter installed in a light source, and
a parallel polarization filter having a direction parallel to the polarization direction of the first polarization filter;

detecting a bright spot from the parallel polarization image; and

detecting a pupil from the orthogonal polarization image.

(9)

A program that causes a computer to function as a control unit to perform:

an operation of obtaining a parallel polarization image and an orthogonal polarization image from a sensor that includes a second polarization filter, the second polarization filter including
an orthogonal polarization filter having a direction perpendicular to a polarization direction of a first polarization filter installed in a light source, and
a parallel polarization filter having a direction parallel to the polarization direction of the first polarization filter;

an operation of detecting a bright spot from the parallel polarization image; and

an operation of detecting a pupil from the orthogonal polarization image.

### REFERENCE SIGNS LIST

1 gaze estimation system
11, 11a infrared light source
12 polarization filter
13 imaging device
14 polarization filter
15 light path separating device
16 PD element
17 light shield
100 gaze estimation arithmetic device
110 control unit
111 parallel polarization image obtaining unit
112 bright spot detecting unit
113 orthogonal polarization image obtaining unit
114 pupil type determining unit
115 pupil position detecting unit
116 gaze estimating unit
120 memory unit

1. An information processing device comprising:
a light source that includes a first polarization filter;
a sensor that includes a second polarization filter; and
a control unit that processes an image obtained by the sensor, wherein
the second polarization filter includes
an orthogonal polarization filter having a direction perpendicular to a polarization direction of the first polarization filter, and
a parallel polarization filter having a direction parallel to the polarization direction of the first polarization filter, and
the control unit
detects a bright spot from a parallel polarization image obtained by the sensor, and
detects a pupil from an orthogonal polarization image obtained by the sensor.

2. The information processing device according to claim 1, wherein the control unit estimates gaze information based on a center position of the pupil and a center position of the bright spot.

3. The information processing device according to claim 2, wherein the control unit
detects, as the bright spot, a first Purkinje image from the parallel polarization image, and
detects, as the pupil, fundus reflex light from the orthogonal polarization image.

4. The information processing device according to claim 3, wherein, from among pixels of the sensor, the number of parallel polarization pixels corresponding to the parallel polarization filter is greater than the number of orthogonal polarization pixels corresponding to the orthogonal polarization filter.

5. The information processing device according to claim 4, wherein pixels of the sensor are formed with an arrangement in which the orthogonal polarization pixels, which are disposed distantly, are individually surrounded by a plurality of the parallel polarization pixels.

6. The information processing device according to claim 2, wherein the control unit
transforms a center position of the detected bright spot to relative coordinates normalized with an image size of the parallel polarization image,
transforms a center position of the detected pupil to relative coordinates normalized with an image size of the orthogonal polarization image, and
estimates the gaze information based on each set of the normalized relative coordinates.

7. The information processing device according to claim 1, wherein the control unit
determines, based on a bright spot detection result obtained from the orthogonal polarization image and the parallel polarization image, whether a bright pupil or a dark pupil is obtained, and
when a dark pupil is obtained, inverts luminosity of the orthogonal polarization image and then detects the pupil.

8. An information processing method implemented in a processor, comprising:
obtaining a parallel polarization image and an orthogonal polarization image from a sensor that includes a second polarization filter, the second polarization filter including
an orthogonal polarization filter having a direction perpendicular to a polarization direction of a first polarization filter installed in a light source, and
a parallel polarization filter having a direction parallel to the polarization direction of the first polarization filter;
detecting a bright spot from the parallel polarization image; and
detecting a pupil from the orthogonal polarization image.

9. A program that causes a computer to function as a control unit to perform:
an operation of obtaining a parallel polarization image and an orthogonal polarization image from a sensor that includes a second polarization filter, the second polarization filter including
an orthogonal polarization filter having a direction perpendicular to a polarization direction of a first polarization filter installed in a light source, and
a parallel polarization filter having a direction parallel to the polarization direction of the first polarization filter;
an operation of detecting a bright spot from the parallel polarization image; and
an operation of detecting a pupil from the orthogonal polarization image.
